(19) United States
(12) Patent Application Publication
(10) Pub. No.: US 2005/0026293 A1
(43) Pub. Date: Feb. 3, 2005

(54) Plasmids from an extremely thermophilic microorganism and derived expression vectors

(75) Inventors: Birgitte Kiaer Ahring, Horsholm (DK); Anders Clausen, Horsholm (DK); Marie Just Mikkelsen, Bronshoj (DK)

Correspondence Address: MERCHANT & GOULD PC, P.O. BOX 2903, MINNEAPOLIS, MN, 55402-0903, US

(73) Assignee: DTU - Technical University of Denmark, Lyngby (DK)

(21) Appl. No.: 10/778,935

(22) Filed: Feb. 13, 2004

Related U.S. Application Data
(63) Continuation of application No. PCT/DK02/00535, filed on Aug. 13, 2002.
(60) Provisional application No. 60/311,871, filed on Aug. 13, 2001.

(51) Int. Cl. C12N 15/74

(52) U.S. Cl. 435/476

(57) ABSTRACT

The present invention concerns the isolation of plasmids from extremely thermophilic anaerobic microorganisms and their use in genetic transformation of thermophilic and mesophilic microorganisms. More particular the invention concerns the use of thermostable plasmid vectors as tools for creating shuttle vectors for genetic transformation of extremely thermophilic anaerobic microorganisms.

### TECHNICAL FIELD OF THE INVENTION

The present invention concerns genetic transformation of extremely thermophilic anaerobic microorganisms. More particular the invention con thermostable plasmid vectors as tools for genetic transformation of extremely thermophilic anaerobic microorganisms.

### BACKGROUND OF THE INVENTION

Extremely thermophilic anaerobic microorganisms, growing at or above 70° C. have long attracted considerable industrial attention. The high temperature optimum of these organisms, and their capability of converting a variety of simple and complex carbohydrates, make them especially interesting as biocatalyst in a number of industrial processes. Polysaccharide degrading enymes like xylanases, cellulases and amylases with high temperature optimum and high efficiencies have been cloned from thermophilic anaerobic microorganisms. Bio-transformation of agricultural products and waste from agriculture, forestry, industry and municipal sources with thermophilic anaerobic microorganisms under simultaneous production of industrial important compounds such as fuel ethanol, are becoming increasingly interesting areas for industrial production.

Thermophilic conditions will ensure high conversion rates and high temperatures will increase the product formation rate and reduce the risk of contamination. In the case of ethanol, as an example, thermophilic anaerobic microorganisms are potential candidates for bio-ethanol production from agricultural products or from waste. Ethanol can substitute for the octane booster Methyl-Tertiary-Butyl-Ether (MTBE) in car fuel or be used directly as a liquid fuel. In contrast to yeasts and the majority of mesophilic bacteria, the thermophilic anaerobic microorganisms are able to metabolise a wider range of substrates, including complex carbohydrate polymers, to yield ethanol. Since the fermentation catalysed by these thermophiles takes place at temperatures just below the boiling point of ethanol, which is about 90° C., it is easy to strip the ethanol from the bioreactor during production. These advantageous properties of thermophilic anaerobes could be exploited for biocatalysis processes using high efficiency reactors and facilitated product recovery (Lynd, 1989; Weimar et al., 1984). Improvement of product yield and minimisation of by-product formation is, however, still essential in order to make these bio-catalytic processes economically viable on an industrial scale.

The genetic or metabolic engineering approaches needed to optimise product yield and minimise un-wanted by-product formation of anaerobic thermophilic microbial strains, are hampered by the lack of suitable thermostable plasmid-based genetic systems.

Plasmids from Thermophilic Anaerobic Microorganisms

Despite the large number of thermophilic anaerobic microorganisms isolated and studied, only a few plasmids have been identified. The plasmids are listed in Table 1.

All pTA plasmids were isolated from partially described thermophilic clostridia capable of converting a number of carbohydrates into ethanol (Kurose et al., 1989b). From this study only 3 out of 320 strains contained one or two plasmids. The four plasmids which were found ranged in size from 1.5 kb to 7 kb. Restriction maps were constructed from the plasmids. No special phenotypes were found to be associated with these plasmids, and they remain cryptic (Kurose et al., 1989a). No information as to copy number is available. In addition, sequence information for these plasmids is lacking, making these plasmids less attractive as the basis for a genetic system for anaerobic thermophilic microorganisms. The fact that all pTA plasmids have been derived from poorly described isolates means that the characterisation of these plasmids in their natural hosts is hampered by the lack of information concerning the host strains.

Tsoi et al. (1987) reported that plasmids were present in four out of seven Clostridium thermocellum strains from their culture collection. They ranged in size from 25 kb to 50 kb, which initially makes them unattractive for vector construction. In addition no sequence information is available.

In thermophilic anaerobic bacteria fermenting hemicellulose, Weimer et al. (1984) found that four out of seven isolated strains contained one plasmid each. All plasmids were small, 1.9-2.4 kb. The plasmid-containing strains were not classified, but were believed to belong to either Thermoanaerobacterium, Thermoanaerobacter or Thermobacteroides. Determination of plasmid size was done by comparing covalently closed circular plasmid migration patterns to E. coli plasmids. Comparing migration patterns of different covalently closed circular plasmids does, however, not necessarily give any information of the actual plasmid size. Only comparison of linearized forms will give the desired information. The presence of plasmids in these strains did not correlate with any gross morphological or physiological characteristics of the organisms (Weimer et al., 1984). The fact that all pDP plasmids have been derived from scarcely described isolates means that the characterisation of these plasmids in their natural hosts is hampered by the lack of information concerning the host strains. In addition no sequence information is available.

Thermoanaerobacterium saccharolyticum DSM571 was shown to harbour two different plasmids, pNB1 4.9 kb and pNB2 2.0 kb (Belogurova et al., 1991). The copy number of pNB1 is 1-2 per cell. The cells were resistant to streptomycin and kanamycin up to 20 μg/ml. By addition of streptomycin or kanamycin the copy number of pNB1 increased to 5-10 plasmid copies per cell without affecting the growth rate. In addition, hydrogen production was strongly affected by the presence of antibiotics. Kanamycin and streptomycin increased the hydrogen production by almost 100%, whereas other antibiotics (ampicillin, tetracycline, chloramphenicol and cycloserine) inhibited hydrogen production and cell growth completely (Belogurova et al., 1991). pNB2 has been fully sequenced (Delver et al., 1996) whereas only limited sequence information from pNB1 is available. A 400 bp fragment within a Pstl-Hindlll region, found on both plasmids, show 100% homology

TABLE 1
Plasmids found in thermophilic anaerobic bacteria.
Km^{R}, kanamycin resistance Sm^{R}, streptomycin resistance.

| | | | Sequence | | Temperature |
|---|---|---|---|---|---|
| Plasmid | | Functions | information | | Optimum |
| name | Size | associated | available | Isolated from (reference) | (strain) |
| pTA106 | 1.5 kb | Cryptic | No | Thermophilic Clostridia (Kurose et al., 1989a) | 60° C. |
| pTA729 | 1.5 kb | Cryptic | No | Thermophilic Clostridia (Kurose et al., 1989a) | 60° C. |
| pTA688S | 2.3 kb | Cryptic | No | Thermophilic Clostridia (Kurose et al., 1989a) | 60° C. |
| pTA688L | 7.0 kb | Cryptic | No | Thermophilic Clostridia (Kurose et al., 1989a) | 60° C. |
| pNB1 | 4.9 kb | Possibly Km^{R}, | No | Thermoanaerobacterium thermosaccharolyticum DSM 571 | 55-60° C. |
| | | Sm^{R} | | (Belogurova et al., 1991) | |
| pNB2 | 2.0 kb | Possibly Km^{R}, | Yes | Thermoanaerobacterium thermosaccharolyticum DSM 571 | 55-60° C. |
| | | Sm^{R} | | (Belogurova et al., 1991; Delver et al., 1996) | |
| pFA1 | 25 kb | Cryptic | No | Clostridium thermocellum F1 (Tsoi et al., 1987) | 60° C. |
| pFA7 | 25 kb | Cryptic | No | Clostridium thermocellum F7 (Tsoi et al., 1987) | 60° C. |
| pFB1 | 45-50 kb | Cryptic | No | Clostridium thermocellum F1 (Tsoi et al., 1987) | 60° C. |
| PSH49 | 8.0 kb | Cryptic | No | Thermophilic anaerobe FERM 7495 (AGENCY OF IND SCI & | 70° C. |
| | | | | TECHNOLOGY (AGEN), 1985a; AGENCY OF IND SCI & | |
| | | | | TECHNOLOGY (AGEN), 1985f) | |
| PSH29 | 6.1 kb | Cryptic | No | Thermophilic anaerobe FERM 7494 (AGENCY OF IND SCI & | 69° C. |
| | | | | TECHNOLOGY (AGEN), 1985d; AGENCY OF IND SCI & | |
| | | | | TECHNOLOGY (AGEN), 1985c) | |
| PSH62 | 5.3 kb | Cryptic | No | Thermophilic anaerobe FERM 7493 (AGENCY OF IND SCI & | 66° C. |
| | | | | TECHNOLOGY (AGEN), 1985e; AGENCY OF IND SCI & | |
| | | | | TECHNOLOGY (AGEN), 1985b) | |
| pFB7 | 45-50 kb | Cryptic | No | Clostridium thermocellum F7 (Tsoi et al., 1987) | 60° C. |
| pDP5009 | 3.8 kb | Cryptic | No | Not classified bacterium. Possibly Thermoanaerobacterium, | 60° C. |
| | | | | Thermoanaerobacter, Thermobacteroides (Weimer et al., 1984) | |
| pDP5010 | 2.4 kb | Cryptic | No | Not classified bacterium. Possibly Thermoanaerobacterium, | 60° C. |
| | | | | Thermoanaerobacter, Thermobacteroides (Weimer et al., 1984) | |
| pDP5012 | 2.4 kb | Cryptic | No | Not classified bacterium. Possibly Thermoanaerobacterium, | 60° C. |
| | | | | Thermoanaerobacter, Thermobacteroides (Weimer et al., 1984) | |
| pDP5016 | 1.9 kb | Cryptic | No | Not classified bacterium. Possibly Thermoanaerobacterium, | 60° C. |
| | | | | Thermoanaerobacter, Thermobacteroides (Weimer et al., 1984) | |

(Kalyuzhnyi et al., 1992) so it was suggested that the smaller plasmid pNB2 originally has derived from pNB1. More sequence information is needed to elucidate this hypothesis. It is, however, very likely that some kind of recombination event has occurred. The sequence of pNB2 reveals three open reading frames (ORF's). One of the ORF's, ORF289, shows homology to replication proteins encoded by rolling circle (RC) plasmids of the pC194/pUB110 family (Delver et al., 1996). Strongest similarity was found to RC plasmids of the pC194 family, in particular three plasmids showed significant similarity, pTB913, pBC1 and pST1. These plasmids were originally identified in the moderate thermophilic bacteria Bacillus sp., Bacillus coagulans and Streptococcus thermophilus, respectively (Ammendola et al., 1992; Muller et al., 1986; Janzen et al., 1992). Rolling circle plasmids replicate via single stranded DNA (ssDNA) intermediates. ssDNA intermediates of pNB2 were indeed found in T. saccharolyticum (Delver et al., 1996), suggesting that pNB2 actually replicates via the rolling circle mechanism. The majority of rolling circle plasmids containing single stranded origins (ss-origins) are known to be host specific (Gruss et al., 1989), and not surprisingly pNB2 failed to replicate in both E. coli and B. subtilis. In addition, no significant similarity to any known ss-origins from other RC plasmids was found. pNB2 derives from a bacterial strain with a temperature optimum at 60° C., indicating that this organism belongs to the thermophilic microorganisms but not to the extremely thermophilic microorganisms. This means that the plasmid stability will be decreased at temperatures above 60° C. and it is consequently less attractive to use pNB2 at temperatures above 60° C.

Three plasmids, pSH29, pSH49 and pSH62, have been isolated from undescribed thermophilic anaerobic isolates. pSH29 (AGENCY OF IND SCI & TECHNOLOGY (AGEN), 1985d) from isolate FERM 7494 (AGENCY OF IND SCI & TECHNOLOGY (AGEN), 1985c), which has a growth optimum at 69° C. (growth range 50-78° C.). pSH49 (AGENCY OF IND SCI & TECHNOLOGY (AGEN), 1985a) from isolate FERM 7495 (AGENCY OF IND SCI & TECHNOLOGY (AGEN), 1985f), which has a growth optimum at 70° C. (growth range 50-80° C.). pSH62 (AGENCY OF IND SCI & TECHNOLOGY (AGEN), 1985e) from isolate FERM 7493, which has a growth optimum at 66° C. (growth range 50-74° C.). The plasmids range in size from 5.3 kb to 8.0 kb. No information as to the function or DNA sequence of the plasmids is available, and the bacterial isolates are poorly characterised.

Plasmid Constructs Used in Transformation of Thermophilic Anaerobic Bacteria

The first attempts to transform thermophilic anaerobic bacteria were done using the mesophilic plasmids pUB110 and pGS13 as vectors and T. thermohydrusulfuricus as recipient (Table 2) (Soutschek-Bauer et al., 1985).

Transformants were selected on the basis of increased kanamycin and/or chloramphenicol resistance. Plasmid bands were barely visible when attempts were made to recover the plasmids from transformed T. thermohydroslfuricus. These plasmid preparations could however be used to re-transform B. subtilis. One third of the re-transformed B.subtilis clones contained plasmids with increased size. Both plasmids could be stably maintained at 55° C. Initial attemptsto transform T. thermohydrosulfuricus protoplasts failed due tothe sensitivityof this strain to polyethylene glycol (PEG). Therefore, an alternative protocol for transformation of intact cells was developed. The cells are made competent by treatment with Tris-buffer at pH 8.3 followed by DNA uptake in the presence of PEG. Between 2 and 5 μg plasmid DNA was used, resulting in (1-2)×10² transformants. The entire procedure was performed under anaerobic conditions using an anaerobic chamber.

Clostridium thermocellum has been used as recipient in genetic studies by Tsoi et al., 1987. The mesophilic plasmid vectors pHV33 and pMK419 (Table 2) were introduced into C. thermocellum by protoplast transformation. pHV33 and pMK419 are B. subtilis-E. coli shuttle vectors based on pUB110, pC194 and pUC9/pUC19. The protoplasts were stabilized by addition of sorbitol, but the number of regenerated protoplasts remained extremely low. Catalase treatment of the surface of agar plates improved the regeneration process dramatically, eventually resulting in 0.01-10% regeneration. Approximately 7 μg plasmid DNA produced 70 transformants.

TABLE 2
Candidate vector systems for extremely thermophilic anaerobic bacteria. Km^{r} kanamycin resistance,
Cm^{r} chloramphenicol resistance, Tc^{r} tetracycline resistance, Sm^{r} streptomycin resistance,
Em^{r} erythromycin resistance. *Plasmids originally isolated from S. aureus and shown here replicate
in B. subtilis and are in most cases maintained in B. subtilis.

| Plasmid | | Genetic | Gram positive | | | Temperature |
|---|---|---|---|---|---|---|
| name | Size | marker | origin | Hybrid description | Replicates in (reference) | Maximum |
| pUB110 | 4.5 kb | Km^{r} | S. aureus | | T. thermohydrosulfuricus DSM | 55° C. |
| | | | | | 568 (Soutschek-Bauer et al., 1985) | |
| pGS13 | 2.9 kb | Km^{r}, Cm^{r} | S. aureus | PUB110 + pC194 | T. thermohydrosulfuricus DSM | 55° C. |
| | | | | | 568 (Soutschek-Bauer et al., 1985) | |
| pHV33 | | Km^{r}, Tc^{r}, | S. aureus | PUB110 + pBR322 | C. thermocellum F7 (Tsoi et al., | 60° C. |
| | | Ap^{r} | | | 1987) | |
| pMK419 | 5.6 kb | Km^{r}, Ap^{r} | S. aureus | PMK4 (pC194 + pUC9) + pUC19 | C. thermocellum F7 (Tsoi et al., | 60° C. |
| | | | | | 1987) | |
| PCTC1 | 7.2 kb | Em^{r} | S. lactis | PAMβ1 and Gram negative replicon | Ta. thermosaccharolyticum | 60° C. |
| | | | | R2 | (Klapatch et al., 1996) | |
| pIKM1 | 6.3 kb | Km^{r} | B. subtilis | PIMP1 (pIM13 + PUC9) + kan^{r} | Thermoanaerobacterium sp. Strain | 60° C. |
| | | | | from S. faecalis. | JW/SL-YS485 (Mai et al., 1997) | |
| pRUKM1 | 7.2 kb | Km^{r} | B. stearothermophilus | PRP9 + pUC18 + kan^{r} from S. | (Mai et al., 2000) | 60° C. |
| | | | | faecalis. | | |
| pUXK | 7.8 kb | Km^{r} | Integration vector | PUC19 + kan^{r} from S. faecalis + | (Mai et al., 2000) | 60° C. |
| | | | | xynA | | |
| pCL1 | 11.9 kb | Cm^{r} | Thermophilic | PTA688L + pBR328 (Cm^{r}) | Thermophilic Clostridium | No |
| | | | Clostridium | | (Kurose et al., 1989a) | Transformation |
| pCS1 | 7.2 kb | Cm^{r} | Thermophilic | pTA688S + pBR328(Cm^{r} | Thermophilic Clostridium | 60° C. |
| | | | Clostridium | | (Kurose et al., 1989a) | |

The transformation experiments done by Soutschek-Bauer and Tsoi were all based on plasmids from Gram-positive mesophilic microorganisms. This type of plasmids is often referred to as Gram positive replicons. Stability of the plasmids decreased with increasing temperature, and the plasmids became difficult to recover.

The first report using a replicon originally isolated from an extremely thermophilic organism came from Kurose et al. (Kurose et al., 1989a). Thermophilic Clostridia were screened for plasmids and these plasmids were eventually used for the development of thermophilic Clostridia-E. coli shuttle vectors. The plasmids pCL1 11.2 kb and pCS1 7.2 kb (table 2) were introduced into a poorly described thermophilic Clostridium by PEG-mediated transformation of intact cells. 1 μg of plasmid pCS1 produced approximately 100 chloramphenicol resistant colonies. pCS1 could be recovered from 60° C. cultures and visualized on agarose gels. Based on restriction enzyme digestion profiles, these recovered plasmids were apparently identical to the original plasmid. The transformants were, however, unstable, and the resistance towards chloramphenicol was easily lost upon repeated cultivation. Stability of the transformants was highly dependent on the choice of recipient. The plasmid pCL1 failed to produce any chloramphenicol resistant colonies and consequently no transformation had taken place.

The mesophilic plasmid pCTC1 (Table 2), based on pAMβ1 and the Gram-negative R2, replicon was used to electrotransform Thermoanaerobacterium thermosaccharo-lyticum (Klapatch et al., 1996). Prior to electroporation the cells were washed in ice-cold water and re-suspended in 20% glycerol. The transformation efficiency was 5 transformants/10 μg DNA. When pCTC1 was prepared from transformed Ta. thermosaccharolyticum the transformation efficiency increased to 52 transformants/μg DNA. Two liters of cell culture were used to recover plasmids from transformed cells. The extracted plasmid-preps were used in Southern blotting experiments to verify transformation. In addition, E. coli was re-transformed with Ta. thermosaccharolyticum extracted pCTC1. Plasmids from E. coli were characterized by restriction enzyme digestion and found to be identical to the original plasmid. Plasmid stability decreased with increasing temperature and no plasmid bands were detectable at 60° C.

pIKM1, pRUKM1 and pUXK (integration vector) (Table 2), containing the thermostable kanamycin cassette from S. aureus, was used to electro-transform Thermoanaero-bacterium sp. strain JW/SL-YS485 (Mai et al., 1997; Mai et al., 2000). The plasmids could be maintained under selective pressure at 60° C. Transformation frequencies varied between 10 and 100 transformants/μg DNA, and apparently no consistent or significant change in transformation efficiency was observed when electro-poration parameters were varied over a wide range. One litre of cell culture was used to recover the plasmids. Recovered plasmids could be visualised on agarose gels and used to re-transform E. coli. Recovered and re-transformed plasmids were identical to the original plasmids, as determined by restriction enzyme digestion. However, plasmid recovery was difficult and large volumes were essential in order to visualise recovered plasmids, suggesting that the plasmids were present in low numbers at elevated temperatures.

Transformation of thermophilic anaerobic microorganisms has been conducted with mesophilic plasmids only, with the exception of two plasmids (pCL1 and pCS1). They are all difficult to recover and they are quite unstable, and even the thermostable plasmids were only present in low numbers when the host cells were cultivated at elevated temperatures. The highest temperature reported for recovery of plasmids from transformants is 60° C.

Since no sequence information is available for the thermostable plasmids used in these reported transformation experiments, their use in the construction of shuttle vectors must be based solely on their restriction maps. This means that shuttle vector design can not take account of regions of the plasmid with potential importance for replication and copy number control, and their usefulness is thus largely a question of chance.

### SUMMARY OF THE INVENTION

The invention provides plasmids and shuttle vectors, capable of self-replication in extremely thermophilic anaerobic micro-organisms. The invention thereby facilitates the establishment of genetic systems for these industrially important micro-organisms. The new plasmids were isolated following a plasmid screen of a number of extremely thermophilic anaerobic microbial strains. These new plasmids, designated pBAS2, 3653 bp (FIG. 1, pBAS 1863 bp (FIG. 2) and pBAL 8294 bp (FIG. 3) were all isolated from the extremely thermophilic anaerobic microorganism, Anaerocellum thermophilum DSM6725,which has a temperature optimum of 72-75° C. All threeisolated plasmids, namely pBAS2, pBAS and pBAL have been characterized by DNA sequencing, that has shown that each of these plasmids is new isolates. The DNA sequence of pBAS showed it to be a sub-species of pBAS2, whereby nucleotide sequence 1-1863 of pBAS corresponds to nucleotide sequence 638-2501 of pBAS2. Both pBAS2 and pBAL contain ORFs encoding proteins which show homology to known proteins of potential industrial interest. Thus proteins encoded by ORF 11 and ORF 61 of pBAS2 (FIG. 1) show homology to a recombinase protein and a replication protein, respectively. In pBAL the ORFs 31, 33 and 34 encode proteins showing homology to a DNA poly-merase/DNA repair protein, a sigma factor, and a replication protein, respectively.

The present invention provides isolated and sequenced plasmids, pBAS2, pBAS and pBAL, originating from extremely thermophilic microorganisms which are stable at growth temperatures of at least up to 75° C. and are thus ideally suited for genetic expression systems intended to operate at high temperatures.

A thermostable shuttle vector, pEAKS (FIG. 4) has been constructed from a fusion of pBAS and pBluescript SK+/KanR, that can be transformed into thermophilic anaerobic kanamycin-sensitive strains, conferring kanamycin resistanceThe pEAKS vector includes replication origins, to facilitate propogation in both extreme thermophilic microbial hosts as well as E. coli, two drug resistance markers for selection of transformants and a multiple cloning site, and thereby provides a new and useful genetic system for enhancing the industrial utility of extreme thermophilic anaerobic microorganisms.

Further, the invention provides an extremely thermophilic host cell transformed with the pEAKS vector, which is stably maintained during growth at 70° C.

Since pBAS2, pBAS and pBAL are derived from an extremely thermophilic anaerobic microorganism with a temperature optimum at 72-75° C., they are the most thermostable plasmids ever isolated from extremely thermophilic anaerobic microorganisms.

Knowledge of the DNA sequence of the plasmids pBAS2, pBAS and pBAL greatly facilitates their used in the construction of various vectors. In addition b pBAS2, pBAS and pBAL are small plasmids, and thus well suited for the construction of genetic systems for extremely thermophilic anaerobic microorganisms.

Expression vectors can be constructed from the pBAS2, pBAS or pBAL plasmids provided by the invention by inserting heterogenous protein expression cassettes into the vector sequence. The expression vectors can be transformed into appropriate host cells for the production of heterogenous proteins, which are recovered and subsequently applied for industrial processes at temperatures of 60-75° C. Host cells, which have acquired new catalytic properties following transformation with the expression vector, may also be used directly in biological processes at temperatures of 60-75° C.

Furthermore the yield and/or stability of thermophilic proteins may be enhanced by expression at high temperatures. In cases where it is desired to screen variants thermophilic proteins for improved catalytic properties, the expression vectors and host cells provided by the invention have particular utility, since the expressed proteins can be screened in vivo.

### DEFINITIONS

PCR

Polymerase Chain Reaction. In vitro amplification of DNA fragments.

Cloning

Construction of a recombinant DNA molecule and amplification of this molecule in Escherichia coli.

Restriction Enzyme

Restriction enzymes recognise specific sequences on DNA molecules and cuts/digests the DNA molecule into smaller fragments.

Restriction Map

Physical map of a plasmid based on the location of restriction sites using different restriction enzymes.

Transformation

Introduction of foreign DNA, encoding a new phenotype, into a cell. Selection of viable cells expressing the new phenotype.

PEG Transformation

The use of the chemical polyethylene glycol to media transformation.

Electro-Transformation

The use of electric fields to mediate transformation.

Protoplast-Transformation

Partial removal of the outer cell wall to facilitate transformation.

Open Reading Frame (ORF)

Putative protein encoding sequence on DNA molecules.

Plasmid

Self-replicating extrachromosomal genetic element.

Vector

A vector is identical to a plasmid. The term vector is used to emphasise that the plasmid is used for genetic engineering.

Plasmid Replication

Multiplication of plasmids inside the cell.

Rolling Circle Plasmid

Replication mode of some circular plasmids. A nick is introduced in one of the DNA strands. The free end serves as template for DNA replication, while the plasmid continues to unwind. Replication proceeds until the entire plasmid has been replicated.

Theta Plasmid

Replication mode, where no nicks are introduced. The two DNA strands separate and the free end(s) is used as template for DNA replication, while the two strands continue to separate.

Mesophilic Microorganisms

Microorganisms with temperature optimum for growth from 20-37° C.

Moderate Thermophilic Microorganisms

Microorganisms with temperature optimum for growth from 37-50° C.

Thermophilic Microorganisms

Microorganisms with temperature optimum for growth from 50-65° C.

Extremely Thermophilic Microorganisms

Microorganisms with temperature optimum for growth from 65-80° C.

Single Stranded DNA (ssDNA)

DNA molecule where the double stranded DNA has separated into single strands.

Origin

Section of plasmid that is mandatory for replication.

ss-Oriqgin

Replication initiates at a single stranded DNA sequence within the replication origin, after the two DNA strands have separated.

Cryptic Plasmid

Plasmid with no known function.

Plasmid-Preps

Extraction of plasmid DNA from transformed cells.

Similarity

Relationship between DNA or protein molecules not necessarily derived from a common background.

Replication Initiation

Physical location on the plasmid where replication ini-tiates.

Replication Terminus

Physical location on the plasmid where replication ter-minates.

Genetic Engineering

Introduction of foreign DNA into a cell.

Metabolic Engineering

Introduction of foreign DNA into a cell with the purpose of altering the metabolism of said cell.

Genetic System

Tools to introduce and stably maintain foreign DNA into a cell.

Transformation Efficiency

Number of transformed cells per μg of DNA.

Shuttle Vector

Plasmid, that can be replicated and maintained in two different microorganisms.

Covalently Closed Circular Plasmid

Circular compact topological form of a plasmid, migrating fastest in agarose gels.

Southern Blotting

Sensitive technique to identify small amounts of known DNA in a sample. The DNA sample is applied to a membrane, which is incubated in a defined hybridization solution containing alabelled DNA-probe, complementary to a known DNA sequence.. If the known DNA is present in the sample, then the labelled DNA-probe will recognise it and bind to it. Because the DNA-probe is labelled, for example with a radioactive isotope, it can be visualised by an X-ray autoradiography.

Agarose Gels

Gel of agarose used to separate DNA molecules of different sizes. When an electrical field is applied to DNA embedded in an agarose gel, the DNA migrates according to the length of the DNA molecule. Long DNA molecules migrate slower than small DNA molecules. The migration of DNA molecules in the agarose gel can be visualised and the migration patterns analysed.

DNA Sequencing

Technique to determine the specific sequence of deoxy-ribonucleic acids (DNA) of a DNA molecule.

Heterogenous Protein

Heterogenous protein is a protein encoded by a gene originating from a host cell genome which is heterogenous to the new host in which the protein is expressed. Such proteins include, but are not restricted to, the enzymes alcohol dehydrogenase, carbohydrase, amylase, cellulase, beta-glucanase, beta-glucosidase, alpha-glucosidase, xylanase, oxidoreductase, protease and lipase.

### BRIEF DESCRIPTION OF FIGURES

FIG. 1

Physical map of plasmid pBAS2. Broad arrows indicate open reading frames, where some have been assigned a putative function. Restriction sites for restriction enzymes cutting the plasmid at 3 or less positions are shown on the map. The sub-species plasmid pBAS, nucleotides 1-1863, corresponds to nucleotides 638 to 2501 of pBAS2.

FIG. 2

Physical map of plasmid pBAS. Base pairs are counted clockwise from the EcoRI site. Black arrows indicate open reading frames with no homology to known genes. Grey arrows are open reading frames, which have been assigned a putative function.

FIG. 3

Physical map of plasmid pBAL. Base pairs are counted clockwise from the EcoRI site. Black arrows indicate open reading frames with no homology to known genes. Grey arrows are open reading frames, which have been assigned a putative function.

FIG. 4

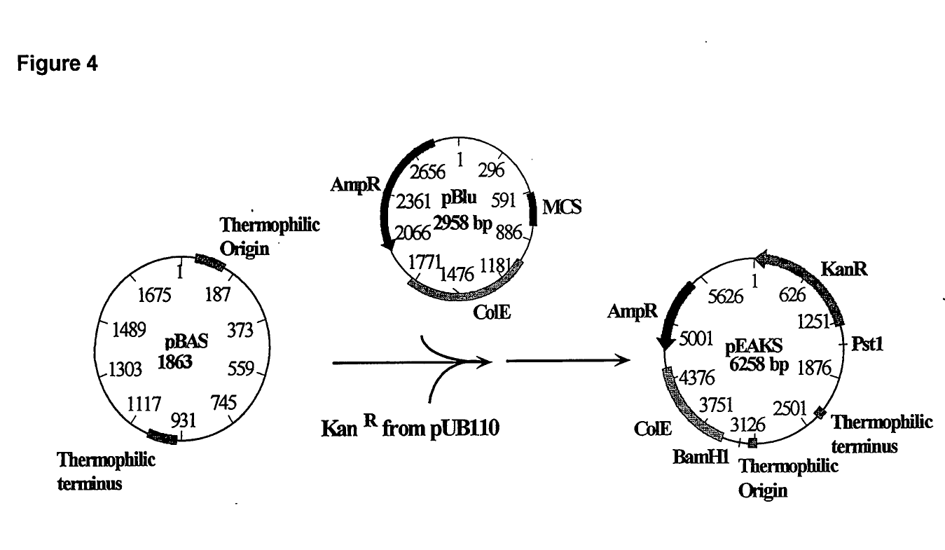

Kanamycin resistance gene was obtained from plasmid pUB110, originally found in a Staphylococcus aureus strain. The resulting thermostable shuttle vector pEAKS contains replication origin from the E. coli vector pBluescript SK⁺ and replication origin from the thermostable plasmid pBAS. As selective markers ampicillin resistance expressed in E. coli and kanamycin resistance expressed in thermophilic anaerobic microorganisms are included.

### DETAILED DESCRIPTION OF THE INVENTION

Anaerocellum thermophilum DSM6725 is a strict anaerobic microorganism with a temperature optimum at 72-75° C. which is freely available from a public culture collection at DSM—Deutsche Sammiung von Mikroorganismen und Zelikulturen GmbH, Mascheroder Weg 1b, D-3300 Braunschweig, Germany, under the accession number DSM6725.

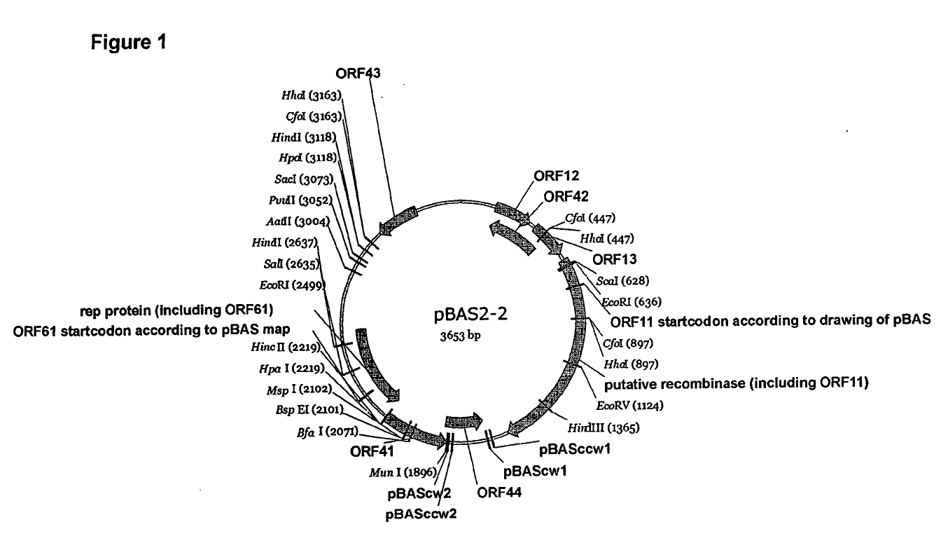

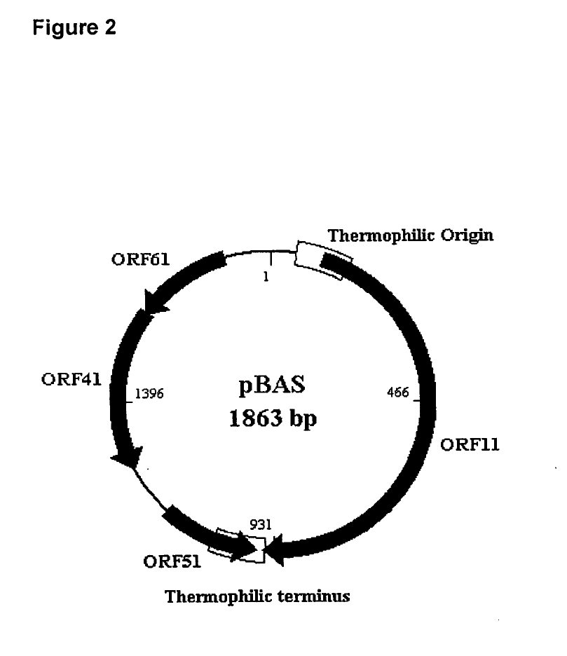

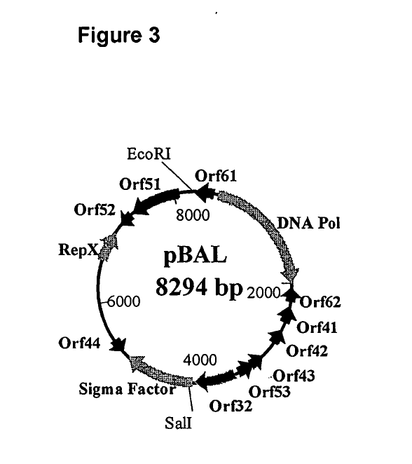

Anaerocellum thermophilum DSM6725 was found to harbour three plasmids, pBAS2, pBAS and pBAL, noner of which have previously been reported. pBAS2 is 3653 bp (FIG. 1) with SEQ. ID NO: 15, pBAS is 1863 bp (FIG. 2) with SEQ ID NO: 22 and pBAL is 8294 bp (FIG. 3) with SEQ. ID NO: 1, and the GC content of the plasmids is 43%, 43% and 39%, respectively.

Sequencing of pBAS2 revealed 8 open reading frames, where ORFs 11 and 61 showed homology to genes encoding a recombinase protein and a replicase protein, respectively. The following ORFs of pBAS2 have been designated the sequence ID numbers: SEQ ID NO: 16=recombinase, SEQ ID NO: 17=replicase (ORF 11), SEQ ID NO: 18=ORF41, SEQ ID NO: 19=ORF42, SEQ ID NO: 20=ORF43, SEQ ID NO: 21=ORF44. Furthermore, a detailed analysis of the DNA structure of pBAS2 revealed a putative replication initiation site and replication terminus.

Sequencing of pBAS revealed that it was a sub-species plasmid (FIG. 2), showing complete sequence identity to a portion of pBAS2, and hence containing only some of the ORFs predicted in pBAS2. Nucleotides 1-1863 in pBAS correspond to nucleotides 638-2501 in pBAS2.

Plasmid pBAL contains 14 open reading frames, of which three showed similarity to known proteins. One ORF showed homology to replication proteins found on various Staphylococcus plasmids. These plasmids, pNVH97, pSK1, pI9789 and pIP680, all replicate via the theta mechanism, indicating that pBAL also employs this type of replication mechanism. Significant homology was also found to sigma factor K from Bacillus thuringiensis and to DNA repair protein from Campylobacter jejuni. The ORFs of pBAL have been designated the following sequence ID numbers: SEQ. ID NO: 2=ORF 31 (DNA polymerase); SEQ. ID NO: 3=ORF32; SEQ. ID NO: 4=ORF 33 (sigma factor); SEQ. ID NO: 5=ORF 34 (replicase); SEQ. ID NO: 6=ORF41; SEQ. ID NO: 7=ORF42; SEQ. ID NO: 8=ORF43; SEQ ID NO: 9=ORF44; SEQ. ID NO: 10=ORF51; SEQ. ID NO: 11=ORF52; SEQ. ID NO: 12=ORF53; SEQ. ID NO: 13=ORF61; SEQ. ID NO: 14=ORF62.

The pBAS2 and pBAL plasmids found in Anaerocellum thermophilum DSM6725, showed no nucleotide sequence similarity to each other. Similarly the proteins encoded by the ORF's in the pBAS2 and pBAL plasmids did not reveal any similarity.

The described plasmids, pBAS2, pBAS and pBAL, are derived from an extremely thermophilic anaerobic microorganism with a temperature optimum at 72-75° C. This makes them the most thermostable plasmids isolated from extremely thermophilic anaerobic microorganisms. pBAS2, pBAS and pBAL are therefore expected to be considerably more thermostable than the plasmids reported by others, since most of the latter plasmids are isolated from cultures with temperature optimum at 60° C.

As opposed to all other plasmids reported, with the exception of pNB2, the full DNA sequence of pBAS and pBAL has been determined. Knowledge of the DNA sequence allows for identification of replication regions and proteins with potential importance for plasmid copy number control, thus providing a far more solid basis for the construction of vectors. In addition both plasmids are small and easy to handle, making them especially useful as the basis for genetic systems for extremely thermophilic anaerobic microorganisms.

The thermostable expression plasmid (pEAKS) is provided according to the invention as an example of several possible expression plasmids. It is to be understood that pEAKS is intended only to be illustrative and in no way limitative. A person skilled in the art will readily understand that once the extremely thermophilic plasmids pBAS2, pBAS and pBAL have been isolated and sequenced, one may derive extremely thermophilic expression plasmids based on these genetic elements.

Marker genes are included in plasmid and shuttle vectors to facilitate the identification and selection of host cells transformed with the vector/plasmid. Marker genes may encode proteins conferring resistance to antibiotics, such as an ampicillin resistance gene, a kanamycin resistance gene, a tetracycline resistance gene, or an erythromycin resistance gene. Alternatively they may encode visual markers, such as those based on beta-galactosidase marker gene expression. Particularly useful marker genes are those which encode proteins which can be actively expressed in the host cell of interest. In the case of an extreme thermophilic anaerobic host cell, it is important to select a marker gene encoding a heat-stable protein. The kanamycin resistance gene is particularly useful in this context, since both the kanamycin resistance protein and the kanamycin antibiotic are rather heat-stable. This is particular important for plasmid stability, where the maintenance of the plasmid in the extreme thermophilic anaerobic host may depend on antibiotic selection pressure. The inclusion of more than one selection marker is particularly useful in shuttle vectors, which should be maintained in two host cell types.

Multiple cloning sites are included in the shuttle vector provided by the invention to facilitate the introduction of additional expression cassettes, which may comprise marker genes or heterogenous protein expression cassettes.

Expression vectors of the invention are used for transformation of host cells, thus constructing microorganisms suitable for industrial production processes at temperatures of 60-75° C. for the manufacture of recombinant proteins which are subsequently recovered. Said proteins can be an alcohol dehydrogenase, a carbohydrase, an amylase, a cellulase, a beta-glucanase, a beta-glucosidase, an oxidoreductase, a protease, an oxidoreductase, or a lipase and they may be applied in a wide variety of industrial processes characterised by high temperatures. Examples of recombinant proteins used in industrial processes are cellulases, amylases, xylanases, β-galactosidases, β-glucosidases, α-glucosidases, and glucanases.

Expression vectors of the invention are also used for transformation of host cells to modify the catalytic properties of the cells, thus improving their performance as biocatalysts in industrial processes at temperatures of 60-75° C. This is known as metabolic engineering and the modified cells may be used for e.g. degradation of biopolymers such as lignocellulosic materials.

Of course the expression vectors of the invention may also be effective at lower temperatures between room temperature and 60° C.

### EXAMPLES

### Example 1

### Screening of Thermophilic Anaerobic Micro-Organisms for Plasmids

Bacterial Strains and Plasmids.

Screening for plasmids was done on thermophilic anaerobic isolates from our strain collection and on DSM6725, which was purchased from DSMZ—Deutsche Sammiung von Mikroorganismen und Zelikulturen GmbH, Germany many.

Media and Culture Conditions.

Anaerobic cultures were grown in a BA medium as previously described (Angelidaki et al., 1990), but the medium was amended with 1 g/l yeast extract (Difco) and cysteine was not added. The medium was reduced with 0.25 g/l sodium sulfide. Appropriate carbon sources, cellulose, glucose, xylose, mannose or galactose was added at 5 g/l and incubation was at 70° C. and pH 6.8.

Plasmid Extraction Procedures.

60 ml overnight cultures were used for extraction of plasmids. Cells were harvested by centrifugation and washed once in 10 ml TE pH 8.0, pelleted and resuspended in TE containing 10 mg/ml lysozyme. The cells were lysed according to the method described by (O'Sullivan et al., 1993),starting from step 2, and the plasmid DNA was subsequently purifed according to standard molecular methods (Ausubel, 1987).

### Example 2

### Isolation and Characterisation of pBAS2, pBAS and pBAL

The plasmids pBAS2, pBAS and pBAL were detected and isolated from Anaerocellum thermophilum DSM6725 as described in example 1.

The presence of the plasmids', designated pBAS2, pBAS and pBAL, in Anaerocellum thermophilum DSM6725 was verified by agarose gel electrophoresis using 1.2 % agarose, stained with ethidium bromide and illuminated by UV light.

Cloning and Sequencing.

For cloning purposes the cloning vector pBluescript SK⁺ and cloning host E. coli DH5-α a was used.

Initially all three plasmids were characterized by digestion with 23 different restriction endonucleases. Restriction endonuclease digests were carried out according to the suppliers recommendation (New England Biolabs). Plasmids pBAS and pBAL showed a unique EcoRI site and were cloned into EcoRI/SAP (shrimp alkaline phosphatase, Boeringer Mannheim) treated pBluescript SK⁺ vector. pBAS was sequenced by “plasmid walking”. In the first round of sequencing the pBluescript KS⁺ primers T3 and T7 were used and the sequence information obtained was then used to design sequencing primers for the next round of sequencing. pBAL revealed an additional unique SalI site, which was used for subcloning into SalI/EcoRI treated pBluescript KS⁺, eventually yielding two plasmids PBMN 4.2 kb and pBAB 4.0 kb. These to plasmids were then sequenced by “plasmid walking”. Restriction mapping and sequencing of pBAS and pBAS2 revealed that pBAS was a plamsid sub-species of pBAS2. Since part of the nucleotide sequence of pBA2 was homologous to pBAS, the sequence of the unknown part of the plasmid was determined according to the following methods. The unknown part of pBAS2 was amplified using the following primers:

pBAScw1: ccccctgcaggctattcttgtggggtagtgcag (SEQ ID NO:23) pBASccw1: ccccctgcagccttgaaacgcaaataaggtcc (SEQ ID NO:24)

This fragment was then sequenced by plasmid walking starting with primers:
pBASeco_ccw1: ccttccacacgagcaggaftcc (SEQ ID NO:25)
pBASeco_cw1: gctttttgattgcaagatggtattgc (SEQ ID NO:26)

The known part of the plasmid, common to pBAS, was verified by restriction enzyme digestion. The nucleotide residues 1-1863 of pBAS correspond to nucleotide residues 638 to 2501 of pBAS2.

FIGS. 1, 2 and 3 show the three plasmids and the complete sequences of pBAS2, pBAS and pBAL are given in SEQ ID NOs 15, 22 and 1, respectively.

### Example 3

### Derivation of pEAKS Expression Vector from pBAS

pBAS was used as the thermostable backbone for construction of a vector replicating in thermophilic anaerobic microorganisms. A 1841 bp fragment of plasmid pBAS, including potential replication region and replication protein was amplified by PCR using the following primer sequences:
pBAS upper1-20: gcgggatccgtcgacgaaftcgaaaagcagataag (SEQ.ID NO:27)
pBAS lower184: gggctgcagtctcgtttaacaactaca (SEQ. ID NO:28)
   thereby introducing unique BamH1 and Pst1 restriction sites in the PCR pruduct. Subsequently the BamH1, Pst1 digested pBAS PCR product was cloned into pBluescript SK⁺/Kan^{R} which was restriction enzyme digested with BamH1 and Pst1. pBluescript SK⁺/Kan^{R} was constructed by introducing the kanamycin resistance gene from pUB110 into the pBluescript vector. The Kan^{R} gene in pUB110 was excised by digestion with the restriction enzymes, Mun1 and Nci1, treated with T4 DNA polymerase, and then ligated into EcoRV digested pBluescript. The resulting thermophilic expression plasmid pEAKS is shown in FIG. 3.

The kanamycin-sensitive, thermophilic anaerobic (Clostridium-like) isolate (Sonne et al. 1993), X7B and the kanamycin sensitive strain Thermoanaerobacter mathranli A3M4 were transformed to kanamycin resistance by the thermostable shuttle vector pEAKS (including the kanamycin resistance gene), by using electroporation and PEG mediated transformation.

Initially transformations were conducted at 55 C and kanamycin resistant colonies were selected. The selected colonies were cultivated at both 55° C. and at 70° C. in selective liquid medium (kanamycin added), without any apparent differences in cell growth rates. Following growth of the transformants in selective liquid medium for 20 generations, they were still stable.

### Example 4

Since both plasmids pBAS2, pBAS and pBAL are small and easy to handle, they are ideal as thermophilic backbone for constructing plasmid vectors for genetic engineering and metabolic engineering of thermophilic microorganisms. For example for improving ethanol yield from thermophilic anaerobic microorganisms, by introduction of additional copies of genes responsible for ethanol formation (e.g. Adh, Alcohol dehydrogenase).

In addition the plasmids pBAS2, pBAS and pBAL can be used as basis for constructing expression vectors for over-expressing industrially important thermostable enzymes e.g. cellulases, amylases, xylanases, β-galactosidases β-glucosidases etc. The shuttle vector, pEAKS, which incorporates pBAS, can be used for the expression of thermostable enzyme. The inducible promoter of the xylA gene from Thermoanaerobacter ethanolicus (Erbeznik et al. 1998), is cloned into the Pst1 or EcoR1site of pEAKS, together with a downstream MCR sequence, and xyl A terminator. A gene of interest, encoding for example an ethanol dehydrogenase, can be cloned into the MCS of the plasmid, and subsequently transformed into the extreme thermophilic anaerobic microbial host. Expression of the heterogenous protein from the gene inserted into pEAKS can be regulated by the level of xylose or glucose added to the medium.

### Example 5

Sequence analysis revealed that pBAL encodes at least three proteins: 1) a DNA polymerase/DNA repair protein 2) a sigma K factor and 3) a replication protein. Their gene products, especially DNA polymerase/DNA repair protein encoded by pBAL can be used in DNA manipulating processes used in molecular biology techniques. For example in the PCR reaction.

### Example 6

This example disclosed the construction of a high copy number shuttle vector based on pBAS2 comprising a kanamycin resistance gene.

The entire pBAS2 plasmid is amplified using pBAScw1 and pBASccw1 primers. The resulting molecule is digested with Pst I restriction enzyme and inserted into the Pst I digested E. coli vector pBluescript SK+. The resulting plasmid is called pBlueBAS2.

pUB110 is digested with Mun I and Nci I and the fragment containing the kanamycin resistance gene is treated with T4 DNA polymerase. The kanamycin resistance casette is then inserted into the EcoRV site of pBlueBAS2.

### Example 7

This example disclosed the construction of a low copy number shuttle vector based on pBAS2 comprising a kanamycin resistance gene.

The entire pBAS2 plasmid is amplified using pBAScw3 and pBASccw3 primers.
pBAScw3: ccccggatccctattcttgtggggtagtgcag
pBASccw3: ccccggatccccttgaaacgcaaataaggtcc

The product is digested with BamH I and inserted into the low copy number plasmid pOU71 digested with BamH I. The resulting plasmid is called p71BAS2.

pUB110 is digested with Mun I and Nci I and the fragment containing the kanamycin resistance gene is treated with T4 DNA polymerase. The kanamycin resistance casette is then inserted into the EcoR I and T4 DNA polymerase treated p71BAS2. The resulting plasmid is called p7IBAS2K.

References

Japanese unexamined published patent application no. JP 60-186287 A, Agency of Industrial Science and Technology (AGEN). New plasmid derived from anaerobic thermophilic bacteria—is used as vector in breeding anaerobic thermophilic bacterial. Sep. 21, 1985a.

Japanese unexamined published patent application no. JP 60-186277 A, Agency of Industrial Science and Technology (AGEN). New microorganism Thermoanaerobacter No. 62—contains new plasmid useful as vector in recombinant DNA experiment. Sep. 21, 1985b.

Japanese unexamined published patent application no. JP 60-186278 A, Agency of Industrial Science and Technology (AGEN). New microorganism Thermoanaerobacter No.29—containing new plasmid characterised by specified restriction end nuclease cleavage map. Sep. 21, 1985c.

Japanese unexamined published patent application no. JP 60-186286 A, Agency of Industrial Science and Technology (AGEN). New plasmid-derived from anaerobic thermophilic bacteria. Sep. 21, 1985d.

Japanese unexamined published patent application no. JP 60-186285 A, Agency of Industrial Science and Technology (AGEN). New plasmid derived from anaerobic thermophilic bacteria—is used as vector in DNA recombination using thermophilic bacteria as most. Sep. 21, 1985e.

Japanese unexamined published patent application no. JP 60-186279 A, Agency of Industrial Science and Technology (AGEN). New Thermoanaerobacter No. 49—containing new plasmid (pSH 49), useful as vector in recombinant DNA experiment. Sep. 21, 1985f.

Ammendola, S., Raia, C. A., Caruso, C., Camardella, L., D'Auria, S., De Rosa, M. & Rossi, M. Thermostable NAD+ dependent alcohol dehydrogenase from Sulfolobus solfataricus: gene and protein sequence determination and relationship to other alcohol dehydrogenases (1992), Biochemistry31, 12514-12523.

Angelidaki, I., Petersen, S. P. & Ahring, B. K. Effects of lipids on the anaerobic digestion and reduction of lipid inhibition upon addition of bentonite (1990), Applied Microbiology and Biotechnology 33, 469-472.

Ausubel, F. M. Current protocols in molecular biology (1987) Ed: F. M. Ausubel, R. Brent, R. E. Kingston, D. D. More, J. G. Seidman, J. A. Smith, K. Stuhl, John Wiley & Sons, Inc.

Belogurova, N. G., Mosolova, T. P., Kalyuzhnyy, S. V. & Varfolomeyev, S. D. (1991), Appl. Biochem. Biotechnol. 27(1),1-8.

Delver, E. P., Belogurova, N. G., Tupikova, E. E., Varfolomeyev, S. D. & Belogurov, A. A. (1996), Molecular & General Genetics 253, 166-172.

Erbeznik M, Dawson K A, Strobel H J. Cloning and characterization of transcription of the xylAB operon in Thermoanaerobacter ethanolicus. (1998), J Bacteriol. 180(5),1103-9

Gruss, A. & Ehrlich, S. D. (1989), Microbiol. Rev. 53(2), 231-241.

Janzen, T., Kleinschmidt, J., Neve, H. & Geis, A. (1992), FEMS Microbiology Letters 74(2-3), 175-180.

Kalyuzhnyi, S. V., Mosolova, T. P., Belogurova, N. G. & Varfolomeev, S. D. Plasmids from thermophilic clostridia (1992), Appl. Biochem. Biotechnol. 34-35, 383-389.

Klapatch, T. R., Guerinot, M. L. & Lynd, L. R. (1996), Journal of Industrial Microbiology 16(6), 342-347.

Kurose, N., Miyazaki, T., Kakimoto, T., Yagyu, J., Uchida, M., Obayashi, A. & Murooka, Y. (1989a), Journal of Fermentation and Bioengineering 68, 371-374.

Kurose, N., Yagyu, J., Ohmori, M., Matsumoto, M., Ohsato, K., Yamada, T., Uchida, M. & Obayashi, A. (1989b), Agri-cultural and Biological Chemistry 53, 3179-3186.

Lynd, L. R. Production of ethanol from lignocellulosic materials using thermophilic bacteria (1989),in A Fiechter (Editor) Advances in Biochemical Engineering/Biotechnology 38, 1-52

Mai, V., Lorenz, W. W. & Wiegel, J. Transformation of Thermoanaerobacterium sp. strain JW/SL-YS485 with plasmid plKM1 conferring kanamycin resistance (1997), FEMS Micro-biology Letters 148(2), 163-167.

Mai, V. & Wiegel, J. (2000), Appl. Environ. Microbiol. 66(11), 4817-4821.

Muller, R. E., Ano, T., Imanaka, T. & Aiba, S. (1986), Mol. Gen. Genet 202(1), 169-171.

O'Sullivan, D. J. & Klaenhammer, T. R. Rapid mini-prep isolation of high-quality plasmid DNA from Lactococcus and Lactococcus spp. (1993), Applied and Environmental Microbiology 59(8), 2730-2733.

Sonne-Hansen, J., Mathrani, I.M., & Ahring, B. K. Xylanolytic anaerobic thermophiles from Icelandic hot-springs (1993) App. Microbio. Biotech.38, 537-541.

Soutschek-Bauer, E., Hartl, L. & Staudenbauer, W. L. Transformation of Clostridium thermohydrosulfuricum DSM 568 with plasmid DNA (1985), Biotechnology Letters 7(10), 705-710.

Tsoi, T. V., Chuvil'skaya, N. A., Atakishieva, Ya. Yu., Dzhavakhishvili Ts. D., Akimenko, V.K., and Boronin, A. M. (1987), Molekulyarnaya Genetika Mikrobiologiya i Virusologiya No. 11, 18-23.

Weimer, P. J., Wagner, W., Knowlton, S. & Ng, T. K. Thermophillic anaerobic bacteria which ferment hemicellulose: characterization of organisms and identification of plasmids (1984), Archives of Microbiology, 138, 31-36.

1. A plasmid comprising a nucleotide sequence selected from the group consisting of SEQ ID NO:1, SEQ ID NO:15 and SEQ ID NO:22, a nucleotide sequence which has more than 80% identity to a nucleotide sequence selected from the group consisting of SEQ ID NO:1, SEQ ID NO:15 and SEQ ID NO:22, and nucleotide fragments of SEQ ID NO:1, SEQ ID NO:15 and SEQ ID NO:22 wherein said fragments comprise at least 20 nucleotides.

2. A plasmid or DNA sequence according to claim 1, wherein said plasmid or nucleotide sequence is isolated from an extremely thermophilic microorganism or a thermophilic microorganism.

3. A plasmid or DNA sequence according to any one of claims 1-2, wherein said plasmid or nucleotide sequence is isolated from Anaerocellum thermophilum.

4. A plasmid according to any one of claims 1-3, which comprises a marker gene.

5. A plasmid according to claim 4, wherein said marker gene is a beta-galactosidase gene.

6. A plasmid according to claim 4, wherein said marker gene is a drug resistance gene.

7. A plasmid according to claim 6, wherein said marker gene is selected from the group consisting of an ampicillin resistance gene, a kanamycin resistance gene, a tetracycline resistance gene, an erythromycin resistance gene, and a chloramphenicol resistance gene.

8. A plasmid according to any one of claims 1-7, which comprises a multiple cloning site.

9. A plasmid according to claim 8, wherein said multiple cloning site comprises a restriction site for at least one restriction enzyme selected from the group consisting of EcoRI, HindIII, SacI, BamHI, XbaI, SalI and PstI.

10. A plasmid according to any of claim 1-9 for use as a shuttle vector, comprising a replication region for use in a secondary host different from the primary thermophilic host from which the plasmids with SEQ ID NOS 1, 15 and 22 have been isolated.

11. A plasmid according to claim 10 wherein the secondary host is a mesophilic microorganism, preferably E. coli.

12. A plasmid according to any of claims 1-11, comprising one or more expression control sequences.

13. Method of obtaining a shuttle vector according to any of claims 10 or 11, comprising:
a) digesting a plasmid according to any of claims 1-9 with one or more restriction enzymes to obtain a fragment of said plasmid comprising a replication region,
b) digesting a plasmid suited for the secondary host with one or more restriction enzymes to obtain a fragment of said plasmid comprising a replication region, and
c) ligating said fragments to obtain a plasmid autonomously replicable in both the primary and secondary host.

14. A plasmid according to claim 11, which has more than 60%, preferably more than 70%, more preferably more than 80% nucleic acid identity to pEAKS.

15. A plasmid according to any one of claims 1-12 or 14, wherein a gene has been inserted which codes for a heterogenous protein.

16. A plasmid according to claim 15, wherein said heterogenous protein is an enzyme.

17. A plasmid according to claim 16, wherein said enzyme is selected from the group consisting of an alcohol dehydrogenase, a carbohydrase, an amylase, a cellulase, a beta-glucanase, a beta-glucosidase, an alpha-glucosidase, a xylanase, an oxidoreductase, a protease and a lipase.

18. A host cell transformed with a plasmid according to any one of claims 1-12 or 14-17.

19. A host cell according to claim 18, which is a thermophilic microoganism.

20. A host cell according to claim 19, which is an extremely thermophilic microorganism.

21. A host cell according to any one of claims 18-20, wherein said host cell is an anaerobic microorganism.

22. A host cell according to claim 18-21, wherein said host cell is a bacterium.

23. A host cell according to claim 22, which is a microorganism selected from the group consisting of Thermoanaerobacter, Thermoanaerobacterium, Thermoanaerobium, Thermoanaerobacteroides, Anaerocellum, Caldicellusiruptor, Clostridium, Bacillus, Thermobacillus, Thermus and Thermotoga.

24. A host cell according to claim 18, which is a mesophilic microorganism, preferably E. coli.

25. Use of a host cell according to any one of claims 18-24, for producing a protein and recovering said protein.

26. Use of a host cell according to any one of claims 18-25, wherein the expressed protein confers said host cell with a changed phenotype.

27. Use of a host cell according to any of claims 18-24 in a process of degradation or fermentation of biomaterial.

28. A protein encoded by any one of the open reading frames of SEQ ID NO:1 oropen reading frames of SEQ ID NO:15.

29. A protein comprising an amino acid sequence selected from the group consisting of SEQ ID NOs:2-14 and 16-20.

30. A protein, characterised by being a variant of the DNA polymerase/DNA repair protein encoded by SEQ ID NO:2, wherein the amino acid identity between said protein and said DNA polymerase is at least 60%, preferably at least 80%, more preferably at least 90%, said protein exhibiting DNA polymerase activity.

31. Use of a DNA polymerase/DNA repair protein of any one of claims 29-30 for the synthesis or repair of DNA.


SEQUENCE LISTING (28 sequences)

SEQ ID NO: 1 - DNA, 8294 bp, Anaerocellum thermophilum
  CDS (339)..(2003): Orf31, DNA polymerase
ttctacaaaggcatagggttaagactaactgcacttaatggcaaaaagccattccttgccgactggtactataaagaactaagcgacgaagagcttttagagtactgggctcagggctataacatcggtgggaagatgggagaaaaaagctgctggatggtggatatcgacatagaccacccagcagcaaacaaaatagttgatttgtaccttccaatagatacgttgaaattcgggcgggcaacgaaacctctatcacatctgctgtatttttcaaaaaattgtaagtcgcttaagagagatttcaaattcagtcaatcagactgaaaaaactaccattgtcgagatccgcagtactggtcaagcagaccatgattccaccttcaatccatccagacactaaagaagaattaagatttgtgggtgaacttaaagaacccttaaaaatcgaagctgatttgctaactgccgctgttaataaaatcgcagctgcgactctaatcggcttgcattggtatgaagggcagcgtcaagattgtgctttagcgctggctggtggtcttttgcgtgctgggtggtctgaagaagaggcagaaaaatttatacaggctgtttgcttagttgcagatgatgaagagtcaaaaaagcggataaaaactgtaattcaaactgctaagaaacaagaggagaacaaggcgaccacaggctggcgtagattggcacaattgataggaaatgaggttgttgcaaaagttattgaatggctggggataaaacaaaccaaagcagagctgctatccatggcagaacttttgagtttggagctgccacctgttgaatttctacttgaaggtcttttgccaaatgaaggccttactatattagcaggcagggaaaagatagggaaaagctggctttgtcttgaacttgccttgagtttagcagcaggaacaggatttctgggcaggcagactaaacgaccttacaaagtgctctatcttgcacttgaagacagcaaaagaagattgcagcaacgaattaaacaattaggaactggcatttcacaaaactgttttgttcttttcaacattgaaagcttacaagaattagaaaagataatcatagaccacaactttgaagtagtgatagtagacaccatgcaagcctttaaaagagctgtaaaactttttacaaacacaaagcttaacacctatgagcaggattataacattagcctaatgttgaacgacattgcaaaaaggcttaattgcacctttataattgtccatcacacaaagaagtcaggcgaactggattttgtaaacgaagtgcttgggagagtaactgcaggtgctgataccatcctgcatttgaccagaggaagaaccgaagctgatggttttctaaaagtcacaggcagagatgttgaagaacaggagcttgcgctgcggtttgaaaatgggcactggtctttgttaggaactgcgaaagaatatgcaatttcgaaagaaaggcgggaaattttagaagtcataaaacaattgggtgggtctgctacaccaaaacaaattgccgaagccttaggaaaaacaagaagcaacgttaacaagctattgctaaaactggctggggatggatatgtgaaggcagtaaatggaatatacagccttgccacagagacagaaaaaagcattcactctattcactctattcactctattcactctattcactctattcactctgtggacagagtgaatagagaagtgaatgagggaacgatggaaaattttagcgttcactctgtagagcccttgagccaactaaatttttcgggggtgagtgaacagagtgaacggagtgaacgcactttattgaaattcgaaaaagataaagttttacattcgacgtttgacttgaaaaaggaagagtggcaggaagcagcagagcaatttaaatgtgagtgcggacacgactttagcatatgccgaagcttttcataaatttgggtgtaggagctgcggaacattatatgctttggtgcaagaagaggatgaaaaaggattgtatattagggtagtaaaagaggaagaggaagctttattgccaataggcagtgaagatttttagaacagtagagcaaaaacagagcaaaaacttgatggcagggcacctgtgaaggtgcccttttgtttttgtccaacaagcagcataacattgcaacattttcaacgctaaaaaaaaacactgatggacaagggacgatatacaagtcgtcctttttttagattgtcaaacgtgtcaaaacagaatttagtgtgacaatgctgcaagggttagctgacaagcacgcaaaagcgcataaaataaagaccagggcaggagagcaaagtgagagcaaaacaagggcaaaagagagttgattttgtactttctatcccagaaagatcagtagaaaaaagtagggaaagtgagcaatttgaatatactggcattctgtatgcaaatctatagcactttttgaaggtgcaatgcagctgtttaaaggtttctaaaacgagcagtgtggacaaaaagtgaaaggtgcagaggagactttctctgcacgaagtagcaacccagcagagaagctctaaagagttagctcatttaaagagcgtggtagtacgaaagtagcacgaaagttaaattcagaggtaataacacaaagcacaaaggcaggggtatagtgcaaaagtgaggttaaagttaggcaaaaaaaaatgacacaaaatggtgattggatgagcagaaacgcaggattttgcattgttacttttgtgacgcttaaaaaaaaggctttgaaatggcgaatgagttacaaagcagcgacccagtagaccaaaaactgccatcgagaaaatgtcaagccaaaaactatgcgaagtttttcacagggcaaaaggaagtaagggaaaattcaaggacgacaagtaagcaaggtgtgaaaaatcggaaacgcagcgtttttgtgaatttcaggagtcaataaaaaaaagagcaagaacgttttaaaggctcatagaacgcagtcaaaggcagggtggtattaaaataccttttgtgctttaatcgctttgctatgggcgtttatgagcgttttagacatgaggtaaaagagaagtaaaagagaagcaaaagaagagtgcagaagggatggattcagtaatacttaacccaaagcaggggatgttgcttcccatccagttgacatccagcaagtgcttgtaactgctgttttgaaaggctttttgtgcgtcaaaaaatgtcaagctttgcgggaattttgtagctgtcatgtcccaaaagtatgcgaggttgcaaggagcaaattatgagcgaaaaaaaagcgcaaaataaacgaggaggcgctttttgattttgtataagccaaaagcttgagagtaggcaatttgaagggcattctaacttggaacaccctacaaggcgtgggtttggaatgcaacattttgcaatgtttaagaaaccttttaacatacaaccagcacttgtggttactgatttggcaggcttttgcgtgttaaaaaatgtttacattttttgaccttttgtcttctcttaaaagcttgtcaatcgtagagaagcctaaagttgcgggctttaggtgtccgcagaatgtaaaatgcgggcaatatgactgggaaagtttaaaacgtgagtttatgcttggagatttcaagactctcaaagagtttgcacaggctaaaaatataagctatggatttttgagaaacaaagcaaaaggctggactcaggagaaaagacaacttagcaaaaccaagagtcagcttgtggttgaaaagacattgcaaaaacaaatagaaaaagcaagcgactacaatactttgcatgttgagttttgggacaggcttcttgacttggtgtggcaggctttgcatgatgagaaaaccatcaaaaccaaggatggcagaataaacatttatgcacttgaaaagctggctttggtggttgagagggtgcaaaaaggacagagacttgctcttggacttgatgagaaaaactcaaatcaaccgcagattgaagacagtattagagaacttatcaaagcacttcatgaaccagatgagccttctgttttaaactaagcttgcaatataaggagtggcaataaacaaatgcagcagtcagaaaaaagacagtttgccaaggtacaaagccagcttttaattgtcgacaacctgaagcttgtgtatcatgttgcaaataaatttatgccatgtccaaagggctattactatgaagttgatgaccttgtcagtgaagggtacattggacttgttgttgcagcaaagaactatgaccctgccaaaggcagtttttcaacctatgcttgcaaggtgattgagagcaagataaaaagaagcttgccaaaatacgagttatcttgtgccatctctttggacagtccagtatcaaaagaacccgaagaagatacatctacagttgcagatataatctcagatggttattcagtcgaaagcgaagtgattaggaaagacacagtccaaaagctacataggtattttgccatcttaaccgaagaggaaaaagaaagagtcttatattacatctccacaggaaagaatttacctgccagtgataatgtgttcaaaaaagcaagaagaaagataatagccaagatgagacaagaacagtttgaggcagaccttgacgacttgacagtgtttatatcctgcccagctgtgcatatagcaagtggtagtaattttggctattcaccagttgaggcaactgttattgatagagaagagaaaagaaaacagcttgaggttttgtctgcaataccacaattggacaagctgagggttttaaaacaacaaggcgaacaggacagaatgaaacttcttgttgccaagtgggaagtagaagagtttatagaacgcaactgggataatctcacaatccagaatgcaaggcttttgcaggactattttgtgtattgtctttcccatctttcaatggtacaaaagtatggcagcaagtacagagagcaaataaaaagagtggtcaaaaagttagctttgtaaaaattttttcttgcgaaaaaagcgaaaaaagcgaaaaaagtcttgagaataggaatatcaaggtttttagtatacctacttttttcgcagctattgcgaaaaaagttcagaaaaagcctaaaaaccttgtggcgactgaattacaaccttgcgaaaaaagtgtgaaaaaagtccatataccctatagggggtataaaactttggtatatccccccaccttgacaagtaggcaagccatcttgagaccggcgggcccagctccaaaaacctgcgattgacacgcacacgaggggggttaaacaaaaaaggttaaaaaacaaacaaaatttcgcattttagcttaaaaagtctgaaataattttaaaattgcttaaaacattgcaattttaaagaggctaaaaagctttcacaggacaaaaaggaaagacagagaacagatacaaaatttcaaaagatcattttgaacctttgaaacattcacagcatagccagtaatttaaatgttcaaattgaaccttataattcctttccagtaattccattcttaccatcgtttataagcacatcaaaatggcaagatggtattttatattcccctatctcattctgcacgttgtagagactttaaaagcgctttgagaaggttgtcaaacgtgtcaaatgcaaagtttaacgtgacaggttgacaatttaacagagctgattttgaccgcagcggtcaaagtggaacaagcttaacaaccatcgagaaaagattaacaggttaacagttttaaattgtcaagaagtgtcaaattgttttataaacgctgcctgtgttaagaaatggtaaggtaagctacagctttgcctctctttaaagcttatcaaccttggcacatctcaaaaatgggcaggtattcaagtttaaccagccgatgaaattcgtggctggtttcaagccaagcccgaccaaattcgatgcttggacatgctaacaaatgctaacctttggagaagacttacaaagggtattctaacttggaacatcctaaatacagggtgtcgaaattcacgacaccccataaactattcggaaatttcggagtccctaaaaaaagagcaacataaaacttacaattttgcagttcctaaaaggtgacttcctaacttggaaagtcattttaaaggggaatggacctataaatccttgtagtttttcaatcaggaccagcgcgctggggtttgcatatgtgaccgcaagttttcaatgtcttttggagcttgtctgttctttgcaggctttagtttaaactccagaattttgagcaaaaagctacttagtaaaacttaaggttcgttaaggttttgaatgcgtagaaaatcagcaaattaaatctaaataccccgaccaaagaaaagccattgaccaagttaaagcgtacatataaactggtttaaagcagtttgcaaacagtttacaagcacgtttacaaagaggtgtaaacatgaaaaacattcaacagttatgcgatgagctgggtatatcgagaacaacagtttataagtacatcagaaggcttggaatagaggtaaagaaggaaggcaatattgcatttataaacgacgatgacattgagaaaataaaagaagcactatcaacagcgagtacaaacagtttgcacacagattacaaacttgagtatatacagtctttacagcagcaaatcgaaacattgcaaaaacaagtagactttttgaaggaacagctcagagcaaaagacgaacagatagccaagctgattcaaaccaatcaaaactttcaggtgcttttaaaagaaaaagaagaacaaatttaccagcttgaaggacaaaaacaaaaaggcagttttttaaaaaagctgtttggccgataagtttttgtttgtcgtgtgccagtataatacagttttgccttttataccgtcgtttataagccacgtctaaaaacagggtagtatttttatatgtatcatcaattagatgcatttctaaaagcctttatgcgcttctgaagggcattttagaaacaaccagcaggggaacatatcaagggtgcgggtagaatagcacctttccagaagggattaaccgcttccagcagcattgaaaaactgaactggcaaataggtaaaaaatagacaaggcagggtaataaatacaaaatggtatccaaaaacatacgaaaagcaccaaaaagaaacaaataaacaaaatcctaattgcacaaaattatatttttgcgcaatcaaaaaatgtttgggtaacacttaaaagtggtgttgattgaaaacaatttttgtgcgtataacaataacagggggtgaaagctatgaaaatgcgcacagtgaacttgagactgccagaagacatttttaatgaaatcggcgagattgcaaagaaaaaagggcttactaaaagcagtctgataagggtaattgttgtggagtatttggaaaagcaaaagaaagaaggtggcaagaatggataatctgccgagggttgaaataccaaaggacaaagagaaaatcaagcaattaatagctgctcttgaacaacaattgcaaaaggacacaaacgaaaaggataaagaaatacacagaaaagctcttgaagatttaaaagccagtttaaacaaaaaagaccacctgtaaaaacttttgttccctatagggttgtgatataataacttacaaagaagataaagacaagtaaaggaggtgcaaagtatgacacagcaaataacaataagagtccctgctaaagaacttgaagaatggcgactcaagattaaagagctttcagacagactggggcatgggaattgctttaagcgaattagtaagacaaggcgttccaaaagcaattgaggaacttgagtcaagaataaaatgagcacgctgtaaattttgtgggcgggacggagacaagcgtgctcattctctttttttaaactttgcttaacttttttcaaaacttttcttagcttttttcaaagaatttgatacattttgtaactctaattttaccacaatggtgcaaaaatgagacaccaaagtttgaacaaaaaacaaaagtagacgttgtaaagattcatgaaaggaaggaggctaaaaaatgcgaaaggacatcagaaagtaccttgaa

SEQ ID NO: 2 - protein, 554 aa, Anaerocellum thermophilum
LSRSAVLVKQTMIPPSIHPDTKEELRFVGELKEPLKIEADLLTAAVNKIAAATLIGLHWYEGQRQDCALALAGGLLRAGWSEEEAEKFIQAVCLVADDEESKKRIKTVIQTAKKQEENKATTGWRRLAQLIGNEVVAKVIEWLGIKQTKAELLSMAELLSLELPPVEFLLEGLLPNEGLTILAGREKIGKSWLCLELALSLAAGTGFLGRQTKRPYKVLYLALEDSKRRLQQRIKQLGTGISQNCFVLFNIESLQELEKIIIDHNFEVVIVDTMQAFKRAVKLFTNTKLNTYEQDYNISLMLNDIAKRLNCTFIIVHHTKKSGELDFVNEVLGRVTAGADTILHLTRGRTEADGFLKVTGRDVEEQELALRFENGHWSLLGTAKEYAISKERREILEVIKQLGGSATPKQIAEALGKTRSNVNKLLLKLAGDGYVKAVNGIYSLATETEKSIHSIHSIHSIHSIHSIHSVDRVNREVNEGTMENFSVHSVEPLSQLNFSGVSEQSERSERTLLKFEKDKVLHSTFDLKKEEWQEAAEQFKCECGHDFSICRSFS

SEQ ID NO: 3 - protein, 192 aa, Anaerocellum thermophilum
MFTFFDLLSSLKSLSIVEKPKVAGFRCPQNVKCGQYDWESLKREFMLGDFKTLKEFAQAKNISYGFLRNKAKGWTQEKRQLSKTKSQLVVEKTLQKQIEKASDYNTLHVEFWDRLLDLVWQALHDEKTIKTKDGRINIYALEKLALVVERVQKGQRLALGLDEKNSNQPQIEDSIRELIKALHEPDEPSVLN

SEQ ID NO: 4 - protein, 310 aa, Anaerocellum thermophilum
MQQSEKRQFAKVQSQLLIVDNLKLVYHVANKFMPCPKGYYYEVDDLVSEGYIGLVVAAKNYDPAKGSFSTYACKVIESKIKRSLPKYELSCAISLDSPVSKEPEEDTSTVADIISDGYSVESEVIRKDTVQKLHRYFAILTEEEKERVLYYISTGKNLPASDNVFKKARRKIIAKMRQEQFEADLDDLTVFISCPAVHIASGSNFGYSPVEATVIDREEKRKQLEVLSAIPQLDKLRVLKQQGEQDRMKLLVAKWEVEEFIERNWDNLTIQNARLLQDYFVYCLSHLSMVQKYGSKYREQIKRVVKKLAL

SEQ ID NO: 5 - protein, 135 aa, Anaerocellum thermophilum
MKNIQQLCDELGISRTTVYKYIRRLGIEVKKEGNIAFINDDDIEKIKEALSTASTNSLHTDYKLEYIQSLQQQIETLQKQVDFLKEQLRAKDEQIAKLIQTNQNFQVLLKEKEEQIYQLEGQKQKGSFLKKLFGR

SEQ ID NO: 6 - protein, 78 aa, Anaerocellum thermophilum
LSTLLVLETFKQLHCTFKKCYRFAYRMPVYSNCSLSLLFSTDLSGIESTKSTLFCPCFALTLLSCPGLYFMRFCVLVS

SEQ ID NO: 7 - protein, 67 aa, Anaerocellum thermophilum
MAVFGLLGRCFVTHSPFQSLFFKRHKSNNAKSCVSAHPITILCHFFLPNFNLTFALYPCLCALCYYL

SEQ ID NO: 8 - protein, 65 aa, Anaerocellum thermophilum
MTATKFPQSLTFFDAQKAFQNSSYKHLLDVNWMGSNIPCFGLSITESIPSALFFCFSFTSLLPHV

SEQ ID NO: 9 - protein, 61 aa, Anaerocellum thermophilum
LFNPPRVRVNRRFLELGPPVSRWLAYLSRWGDIPKFYTPYRVYGLFSHFFRKVVIQSPQGF

SEQ ID NO: 10 - protein, 226 aa, Anaerocellum thermophilum
CLRPAHKIYSVLILFLTQVPQLLLERLVLLIRLKQFPCPSLSESSLILSRHSSSSLAGTLIVICCVILCTSFTCLYLLCKLLYHNPIGNKSFYRWSFLFKLAFKSSRAFLCISLSFSFVSFCNCCSRAAINCLIFSLSFGISTLGRLSILATFFLLLFQILHNNYPYQTAFSKPFFLCNLADFIKNVFWQSQVHCAHFHSFHPLLLLYAQKLFSINTTFKCYPNIF

SEQ ID NO: 11 - protein, 60 aa, Anaerocellum thermophilum
LFLFGAFRMFLDTILYLLPCLVYFLPICQFSFSMLLEAVNPFWKGAILPAPLICSPAGCF

SEQ ID NO: 12 - protein, 79 aa, Anaerocellum thermophilum
MLKGFLNIAKCCIPNPRLVGCSKLECPSNCLLSSFWLIQNQKAPPRLFCAFFSLIICSLQPRILLGHDSYKIPAKLDIF

SEQ ID NO: 13 - protein, 80 aa, Anaerocellum thermophilum
LKNTADVIEVSLPARISTYLLEGTNQLFCLLLGGLCRYPPSSSFFLPSSHRCYSPEPSTLKALRRLVLYSTSRQGMAFCH

SEQ ID NO: 14 - protein, 66 aa, Anaerocellum thermophilum
LYIVPCPSVFFFSVENVAMLCCLLDKNKRAPSQVPCHQVFALFLLYCSKNLHCLLAIKLPLPLLLP

SEQ ID NO: 15 - DNA, 3653 bp, Anaerocellum thermophilum
  CDS (599)..(1588): Putative recombinase
tattgtagtgttatgatctatttcagaagcgaaaagtaaaccgcggtagcgggttactttttgcgtaatatttggaattgtcagtttggcacgggaaatggaatcgggcaggtatagggaaaggcaatagccgtggacggcgttccgattccgctttgtttgtgcgttagcacatgagtcttgctgttatcacgctctgctctgtggtagtacaccgcaaggggtacaccatttggagttttgccgcatctgagagtttagcagtttgcaaaacaaagttcgcagttagaaaagttaggcaaggggcgatgacgtgcacgtctgacacgcttgacgtgcaccgagccgagagtagagcgtgatgacatagaaaaagcccagtagcaggttgtgcgtaccgtgacatgctactgtggaagacaagtaaatagtcaaaaaacggtgcgcttactgttcgtgggctcatgggaataccgtgagcattctggacaggttcaacagcctgaaagggcaaaaccccccttgaaagggtttaaatacacacgtcctttttgtcgttttttgtcggtttaaatatttatgcaggggatgatagggaatgggaaagccgtccataatcaagcaagtactcaatgaattcgaaaagcagataaggttcggggaaagtaagcatgaagcaaagagagaagagcgggagagatgtgaagttactggagagacgtggaatcctgctcgtgtggaaggcatttttagtttctcaacctacagggagtacgttaaggaggcgttagaatttgcgaattgggctcgtactgaaaaggggtgtaaggatttagaacaagcacgggcctatgtgtctgaatatttgcagtcgcatatagacaaagggtatagcgcgtggactgttaagaaagaagcagcagccctggcgaaactgtatcattgtcgtacaactgactttaaagtagagcttcccgcaagacacagggaagagattgagagaagcaggggatacaaagaccacgatagggagtttagcaaagagaggaatagagacattatcatcttttcaaaagctactgggctgagaagaagggaattggaaagagtgagttctcgggatatctttcgtgggcctgacggaagattatatgtgcacgtgagcaacggcaagggcggtagagaaagggatgttcatgttttgcagaaatacgagagagaggttgagaggatagtcagagagcgggaaggaagagacaggctgtttgacagggtccccataaggatggacgtacacagctataggagggaatatgcaagagagcgttacagagaagttgagcgtgagataagtcgtgagagaaagcttttcgacagagttgaggatctcgttcgtagtaggcttacaaggctctatcctgacaggtttagagaaattggcgaaagacaacttactcgtgaactcacaagagctgatgggctttatcatcgcagtgatggtagggagtttgaccgcctggcattgtgggaagtttcaaacgacttgggacataatcgaattgacgttgttgcaagacactatctggattaagcgaataaaggctcaagaaagtggataaaaaaacagggggtatattgatatatcccccctgttttttgtgcgtctacaggaccttatttgcgtttcaaggctattcttgtggggtagtgcagtaaaaaaggttgcctttgtatcttgcctgtagaatcttgccgttttcaaaaacaaattcaacgttgcacttatcagcaagtttcttgattattccctgctcaaattcgtcgaacctgtacccttccattgctcttgctgtgaaataacccagaaggtacattgcttcttcacgtgtaaactcaattgttagctttttcactctcttttaccccctttacgagactgagatattctttgtacaatctgtcaatacgttctgtatcgtcaggttgtacgtttttgagtttttcgtagagattatgaaacaacaggttctctgctgttccaatagggtatttctttctttccgcgtggactagccttttcagttggtttagctttacgttccggatcttgctcagtaggtcttcgacgatattttcaatctgttttgcttcgtcttcttcgatgtagaagcatagttgtttgatttcaagtagcctctgaataagttcctgctttgttaacattccttgtcacccttccccttttcagtcttttttattttgtcgtattctgattcgaactttctcagttggtcagctctgccgtaacgtatgcgtcgaggatttagcatgaatcgtccacggcctattttaatcaacaaatctttctcgattagctttttgattgcaagatggtattgcttttttgacatgtcgatgtttcctttgatgtcgtattcgtatgaaagtgcaaacgtgtagttgttaaacgagattttgtgttgtacaatgtatgagagaattcgcattgctgattttgcaaggtctttatcttccattatgagtttgatagaaatgaatggaagtttgacaaagtctgtgtcttcaaaatcatgtccgatcagtatgaaagtttgtacttcgcctgtttcagggtcgaccatttttttgtaccctatcttcttcatttgaatccccctttttttacctttttccaattctaaagccattataatacacttaaagtaacttgtcaagtaacttcaggtgtaaaaaattacacttcaggtgtaataaattacactttccattcagtattttcaaggctttgtgggtaactttattcttatctatgtatatatcgcctgcgttagcaggcttgaaaatttccagttaggataagcaggaacaacggtcgctgacgctgaacactgacgaaatagctgacgccccaaagtccacaacagtgccaaaccgataacaaaaacatgctaacgcaaacatagactaacgcacgactgacgtcgtgatgtgtgtgtgggcctacctacacacaaaaagaactaacaacagctgactaacgtctgaagagctctaacaacactttgctaacgctgagctaacggacagctcaacgttaacacccgctaaaaaaattagagcaggatttgagcaagaggggcgctgcaaccccctttgaaaccgctttcataacccgaaaagctcttcacccaccgcatcaatattcatacccgcttgcataattattcatccctgattgtaccacaaaacccagaaaagtcctgggaaaatcatgaagattcttccattttccaatttccaggaagcgaattgaaattcccagggattttttgcctttttccagggaatgaactggaatttccagcggtttttccagggacttaaccacacgaaaggtactttccgccatcaaaggcttgcaaacccataggaaactactggcaggcaaaaccttccacccagtagcaaatctcttctaccttgttttgcgagcgttagcagggagctgtccgttagctttgtgttagcatccttctgttagcctttggcacgttagtagacttttgttagttctctcttcttgtttttatcttcattgtgattgttttccagaagactgcttgacattttgt

SEQ ID NO: 16 - protein, 330 aa, Anaerocellum thermophilum
MGKPSIIKQVLNEFEKQIRFGESKHEAKREERERCEVTGETWNPARVEGIFSFSTYREYVKEALEFANWARTEKGCKDLEQARAYVSEYLQSHIDKGYSAWTVKKEAAALAKLYHCRTTDFKVELPARHREEIERSRGYKDHDREFSKERNRDIIIFSKATGLRRRELERVSSRDIFRGPDGRLYVHVSNGKGGRERDVHVLQKYEREVERIVREREGRDRLFDRVPIRMDVHSYRREYARERYREVEREISRERKLFDRVEDLVRSRLTRLYPDRFREIGERQLTRELTRADGLYHRSDGREFDRLALWEVSNDLGHNRIDVVARHYLD

SEQ ID NO: 17 - protein, 163 aa, Anaerocellum thermophilum
MALELEKGKKRGIQMKKIGYKKMVDPETGEVQTFILIGHDFEDTDFVKLPFISIKLIMEDKDLAKSAMRILSYIVQHKISFNNYTFALSYEYDIKGNIDMSKKQYHLAIKKLIEKDLLIKIGRGRFMLNPRRIRYGRADQLRKFESEYDKIKKTEKGKGDKEC

SEQ ID NO: 18 - protein, 109 aa, Anaerocellum thermophilum
MLTKQELIQRLLEIKQLCFYIEEDEAKQIENIVEDLLSKIRNVKLNQLKRLVHAERKKYPIGTAENLLFHNLYEKLKNVQPDDTERIDRLYKEYLSLVKGVKESEKANN

SEQ ID NO: 19 - protein, 99 aa, Anaerocellum thermophilum
MSPRTVSAPFFDYLLVFHSSMSRYAQPATGLFLCHHALLSARCTSSVSDVHVIAPCLTFLTANFVLQTAKLSDAAKLQMVYPLRCTTTEQSVITARLMC

SEQ ID NO: 20 - protein, 70 aa, Anaerocellum thermophilum
MAESTFRVVKSLEKPLEIPVHSLEKGKKSLGISIRFLEIGKWKNLHDFPRTFLGFVVQSGMNNYASGYEY

SEQ ID NO: 21 - protein, 73 aa, Anaerocellum thermophilum
VKKLTIEFTREEAMYLLGYFTARAMEGYRFDEFEQGIIKKLADKCNVEFVFENGKILQARYKGNLFYCTTPQE

SEQ ID NO: 22 - DNA, 1863 bp, Anaerocellum thermophilum
ttcgaaaagcagataaggttcggggaaagtaagcatgaagcaaagagagaagagcgggagagatgtgaagttactggagagacgtggaatcctgctcgtgtggaaggcatttttagtttctcaacctacagggagtacgttaaggaggcgttagaatttgcgaattgggctcgtactgaaaaggggtgtaaggatttagaacaagcacgggcctatgtgtctgaatatttgcagtcgcatatagacaaagggtatagcgcgtggactgttaagaaagaagcagcagccctggcgaaactgtatcattgtcgtacaactgactttaaagtagagcttcccgcaagacacagggaagagattgagagaagcaggggatacaaagaccacgatagggagtttagcaaagagaggaatagagacattatcatcttttcaaaagctactgggctgagaagaagggaattggaaagagtgagttctcgggatatctttcgtgggcctgacggaagattatatgtgcacgtgagcaacggcaagggcggtagagaaagggatgttcatgttttgcagaaatacgagagagaggttgagaggatagtcagagagcgggaaggaagagacaggctgtttgacagggtccccataaggatggacgtacacagctataggagggaatatgcaagagagcgttacagagaagttgagcgtgagataagtcgtgagagaaagcttttcgacagagttgaggatctcgttcgtagtaggcttacaaggctctatcctgacaggtttagagaaattggcgaaagacaacttactcgtgaactcacaagagctgatgggctttatcatcgcagtgatggtagggagtttgaccgcctggcattgtgggaagtttcaaacgacttgggacataatcgaattgacgttgttgcaagacactatctggattaagcgaataaaggctcaagaaagtggataaaaaaacagggggtatattgatatatcccccctgttttttgtgcgtctacaggaccttatttgcgtttcaaggctattcttgtggggtagtgcagtaaaaaaggttgcctttgtatcttgcctgtagaatcttgccgttttcaaaaacaaattcaacgttgcacttatcagcaagtttcttgattattccctgctcaaattcgtcgaacctgtacccttccattgctcttgctgtgaaataacccagaaggtacattgcttcttcacgtgtaaactcaattgttagctttttcactctcttttaccccctttacgagactgagatattctttgtacaatctgtcaatacgttctgtatcgtcaggttgtacgtttttgagtttttcgtagagattatgaaacaacaggttctctgctgttccaatagggtatttctttctttccgcgtggactagccttttcagttggtttagctttacgttccggatcttgctcagtaggtcttcgacgatattttcaatctgttttgcttcgtcttcttcgatgtagaagcatagttgtttgatttcaagtagcctctgaataagttcctgctttgttaacattccttgtcacccttccccttttcagtcttttttattttgtcgtattctgattcgaactttctcagttggtcagctctgccgtaacgtatgcgtcgaggatttagcatgaatcgtccacggcctattttaatcaacaaatctttctcgattagctttttgattgcaagatggtattgcttttttgacatgtcgatgtttcctttgatgtcgtattcgtatgaaagtgcaaacgtgtagttgttaaacgagattttgtgttgtacaatgtatgagagaa

SEQ ID NO: 23 - DNA, 33 bp, Anaerocellum thermophilum
ccccctgcaggctattcttgtggggtagtgcag

SEQ ID NO: 24 - DNA, 32 bp, Anaerocellum thermophilum
ccccctgcagccttgaaacgcaaataaggtcc

SEQ ID NO: 25 - DNA, 22 bp, Anaerocellum thermophilum
ccttccacacgagcaggattcc

SEQ ID NO: 26 - DNA, 26 bp, Anaerocellum thermophilum
gctttttgattgcaagatggtattgc

SEQ ID NO: 27 - DNA, 35 bp, Anaerocellum thermophilum
gcgggatccgtcgacgaattcgaaaagcagataag

SEQ ID NO: 28 - DNA, 27 bp, Anaerocellum thermophilum
gggctgcagtctcgtttaacaactaca